(19) United States
(12) Patent Application Publication
(10) Pub. No.: US 2002/0197228 A1
(43) Pub. Date: Dec. 26, 2002

(54) Skin care kit

(76) Inventors: William Kater LaSala, Mason, OH (US); Ty Eric Martin, Loveland, OH (US); Nancy Coultrip Dawes, Cincinnati, OH (US); Robert Bao Kim Ha, Milford, OH (US); Debra Gay Gehring, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/157,589

(22) Filed: May 29, 2002

Related U.S. Application Data
(60) Provisional application No. 60/294,136, filed on May 29, 2001.

(51) Int. Cl. A61K 7/06; A61K 7/11

(52) U.S. Cl. 424/70.12

(57) ABSTRACT

The present invention relates to a skin care kit comprising a skin care composition contained within a dispenser, capable of consistently delivering a predetermined amount of the skin care composition by actuation of a dispensing package pump wherein said composition is delivered through the dispenser's dispensing surface. The skin care compositions of the present invention are water-in-oil emulsions comprising an oil continuous phase and an aqueous discontinuous phase.

### CROSS REFERENCE TO RELATED APPLICATION

This application claims the benefit of U.S. Provisional Application No. 60/294,136, filed May 29, 2001, and U.S. Ser. No. 09/063,324, filed Apr. 20, 1998.

### TECHNICAL FIELD

The present invention relates to the field of kits comprising conditioning skin care compositions and dispensers for such compositions to encourage consumer use.

### BACKGROUND

Consumers today are faced with an enormous selection of cosmetic and skin care products promising countless skin health and skin care benefits. Such skin care products are available in a number of forms, including creams, lotions, and serums. Also, it is understood in the art that there is a complicated relationship between a particular product's marketing/packaging, its benefits (both real and perceived), and its consumer appeal. Particularly, consumers are often predisposed to purchase the types of packages that are typically associated with their preferred product form. For example, cream formulations are normally associated with a jar while lotions are associated with a dispenser which limits the quantity of composition dispensed per use. Ease of delivery of the product in amounts that provide ease of application and optimum dosing for effectiveness cannot be underestimated in driving consumer preference for one product over another. Lastly, aesthetic benefits unrelated to actual skin health and/or care benefits are a strong factor in the consumer's products purchasing decision. For instance, when a consumer is faced with what appear to be a large selection of similar products, such as a moisturizing composition, the consumer will often select a product based on factors such as packaging, advertising campaigns, fragrance, and brand.

In the skin care market, consumers have developed preferences for particular product forms and correspondingly have developed preferences for the types of packages associated with their preferred product form. Therefore, it is important for those introducing new formulations and/or product forms to carefully consider the packaging choices to ensure an overall product that will be consumer acceptable as well as provide the intended product benefits.

### SUMMARY

The present invention relates to a skin care kit comprising a skin care composition contained within a dispenser, capable of consistently delivering a predetermined amount of the skin care composition by actuation of a dispensing package pump wherein said composition is delivered through the dispenser's dispensing surface. The skin care compositions of the present invention are water-in-oil emulsions, including those comprising silicone elastomers, wherein oil is the continuous phase and water is primarily the discontinuous phase.

### BRIEF DESCRIPTION OF THE DRAWINGS

Alternative embodiments of the dispenser component of the invention shall now be described by way of example with reference to the accompanying drawings, wherein:

FIG. 1 shows a longitudinal sectional view of a dispenser according to a first embodiment of the pump dispenser of the invention;

FIG. 2 shows longitudinal sectional view of a dispenser according to a second embodiment of the pump dispenser of the invention;

FIG. 3 shows a longitudinal sectional view of a headpiece of a dispenser according to a third embodiment of the invention;

FIG. 4 shows a longitudinal sectional view of a headpiece of a dispenser according to a fourth embodiment of the invention; and

FIG. 5 shows a longitudinal sectional view of a headpiece of a dispenser according to a fifth embodiment of the invention.

### DETAILED DESCRIPTION

All percentages and ratios used herein are by weight of the total composition, and all measurements made are at 25° C., unless otherwise designated.

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

All publications cited herein are hereby incorporated by reference in their entirety. The term “topical application”, as used herein, means to apply or spread the compositions of the present invention onto the surface of the skin.

The term “dermatologically-acceptable,” as used herein, means that the compositions or components thereof so described are suitable for use in contact with human skin without undue toxicity, incompatibility, instability, allergic response, and the like.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound medical judgment.

Active and other ingredients useful herein may be categorized or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore, classifications herein are made for the sake of convenience and are not intended to limit an ingredient to the particularly stated application or applications listed.

It has now been discovered that consumers often select compositions that can be delivered in exacting doses in order to optimize exotic and, or expensive skin care active ingredients in the compositions as well as to avoid having to remove excess amounts of the composition from the skin. For example, when desiring a precise amount of a composition to be delivered, consumers usually prefer the controlled dispensing of predetermined amount by using a pump dispenser or other unit dose dispenser in contrast to ajar or tube. The dispenser of the present invention thus is capable of dispensing a predetermined amount on full actuation since the dispenser mechanically controls the quantity of composition to be dispensed from the container. It is also discovered that packages that dispense product onto the actuation surface and having exacting dosing tolerances are popular since the surface the product is dispensed upon acts like a palate wherein the user can selectively dab their fingers into said composition to administer the composition evenly in the amounts desired at specific spots on the skin.

In addition to the group of water in oil emulsions that can be used in the present invention, an alternative water-in-oil emulsion comprises silicone elastomers. When used within a dispenser, capable of dispensing a predetermined amount of composition, improved skin care benefits, including but not limitecd to skin conditioning and the regulation of a skin's condition is realized by the consumer. Without being limited by theory, it is believed that the improved benefits are the result of a synergistic combination of two factors: (1) a composition containing both a water-in-oil emulsion and a silicone elastomer provides for improved skin feel and (2) the dispenser which is capable of dispensing a predetermined, optimal amount of the product to meet the needs of all users.

Another embodiment of the present invention comprises a skin care composition contained within a dispenser such that the composition comprises a water-in-silicone emulsion as follows: from about 25% to about 75% of a hydrophobic phase comprising a silicone oil; from about 0.5% to about 3% of a silicone elastomer; a hydrophilic water phase; from 0% to about 2% of a dimethicone copolyol emulsifier; from about 0.1% to about 10% of a reflective particulate material; and from about 0.0001% to about 20% of a skin care active, wherein the composition has a viscosity of between from about 15000 cps to about 100,000 cps and a pH of from about 5 to about 7.

Silicone elastomer containing water-in-oil compositions impart to the skin a more luxurious, silky feel upon application than traditional oil-in-water emulsions and require more massaging time to fully absorb the product into the skin. The additional massage time leaves the consumer with a feeling of pampering, adding to the prestige perception.

The compositions of the invention are useful for topical application and for also providing skin conditioning, including moisturization following application of the composition to the skin. More particularly, the compositions of the present invention are useful for regulating skin condition, including regulating visible and/or tactile discontinuities in skin, including but not limited to visible and/or tactile discontinuities in skin texture and/or color, more especially discontinuities associated with skin aging. Such discontinuities may be induced or caused by internal and/or external factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin.

The present invention also relates to methods of regulating skin condition by topical application of the present skin care compositions contained therein using the dispensing devices previously mentioned.

The skin care kits of the present invention provide additional benefits, including stability, absence of significant (consumer-unacceptable) skin irritation and good aesthetics.

The skin care kits of the present invention comprise a skin care composition contained within a dispenser. The skin care composition is comprised of a water-in-oil emulsion and alternatively a water in oil emulsion containing a silicone elastomer.

The skin care kits herein may also include a wide variety of other ingredients. The skin care kits of the present invention, are described in detail hereinafter.

I. Skin Care Composition

The skin care compositions of the present invention comprise water-in-oil emulsions known to those skilled in the art and disclosed in PCT Applications WO 00/62743, published Oct. 26, 2000 and WO 02/05952, published Jan. 17, 2002.

Water-In-Oil Compositions

The compositions of the present invention comprise a water-in-oil emulsion, preferably a water-in-silicone oil emulsion, within which the other components of said emulsion are incorporated to enable delivery of the skin-benefiting components to the skin at an appropriate concentration. The emulsion can thus act as a diluent, dispersant, solvent, or the like for the other composition components which ensues that the composition can be applied to and distributed evenly over the selected target at an appropriate concentration.

Suitable water-in-oil emulsions include conventional or otherwise known carriers that are dermatologically acceptable. The emulsion components should also be physically and chemically compatible with the essential components described herein, and should not unduly impair stability, efficacy or other use benefits associated with the compositions of the present invention.

Such water-in-oil emulsions comprise a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, to form a hydrophilic dispersed phase and a hydrophobic continuous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase.

The composition of the present invention comprises water-in-oil emulsion and alternatively water-in-oil emulsions comprising silicone elastomers. Such compositions include those having an apparent viscosity of from about 15,000 to about 100,000 centipoise (cps). These water-in-oil compositions may also include skin care actives that are solubilized either into the water or discontinuous phase and that is ultimately dispersed into the oil or continuous phase of the composition or in the oil phase. Among the skin care actives useful in the compositions of the present invention are niacinamide, vitamin E acetate, dexpanthenol, palmitoyl-pentapeptides, salicyclic acid, retinoids, sunscreens, and mixtures thereof. Non-limiting examples of compositions useful herein include:

a) a thin serum having a viscosity of from about 15,000 to about 40,000 cps, alternatively about 25,000 cps, b) a lotion having a viscosity of from about 25,000 to about 100,000 cps, alternatively about 40,000 cps, and c) a cream having a viscosity of from about 25,000 to about 100,000 cps, alternatively about 60,000 cps.

Viscosity Determination

Viscosity can be determined using a Brookfield RVDV-II digital viscometer, a T-C spindle (Spindle 93, 27.1 mm crossbar length), at 5 rpm, or the equivalent thereof. Prior to viscosity measurement, the composition is allowed to stabilize following its preparation or any agitation which results from handling. Generally, stabilization should last at least 24 hours under conditions of 25° C. +/−1° C. and ambient pressure. In further preparation for viscosity measurements, the compositions are placed in containers which will produce no or only minimal frictional effects on the viscosity determination (e.g., a 2 oz. glass jar with an orifice of at least 28 mm). The viscosity is measured with the composition at a temperature of 25° C. +/−1° C. and after 30 seconds of spindle rotation. Five (5) viscosity measurements are gathered and the mean of the measurements is calculated in order to determine the viscosity of the composition.

The compositions of the present invention generally have a pH of from about 3 to about 9, more preferably about 4 to about 8, even more preferably about 5 to about 7, and most preferably about 6.25 to about 7.

Water-in-Oil Emulsion

1. Hydrophobic Phase

Emulsions according to the present invention contain a hydrophobic phase comprising a lipid, oil, oily or other hydrophobic component. The compositions of the present invention preferably comprise from about 25% to about 90%, preferably from about 27% to about 80%, and more preferably from about 30% to about 70% by weight of the composition, of a hydrophobic phase. The hydrophobic component may be derived from animals, plants, or petroleum and may be natural or synthetic (i.e., man-made). Preferred hydrophobic components are substantially water-insoluble, more preferably essentially water-insoluble. Preferred hydrophobic components are those having a melting point of about 25° C. or less under about one atmosphere of pressure, and are suitable for conditioning the skin.

Nonlimiting examples of suitable hydrophobic components include those selected from the group consisting of:

a) Silicone Elastomer

The compositions of the present invention may include from about 0% to about 30%, by weight of the composition, of a silicone elastomer component. Alternatively, the composition includes from about 0.1% to about 30%, or from about 0.5% to about 10%, by weight of the composition, of a silicone elastomer component All such percentages as they pertain to the silicone elastomer are based on the amount of elastomer, not the carriers or by-products that may be included in commercially available materials. Commercially available silicone elastomers are often introduced into the overall composition in solution with a silicone oil. Such silicone oil amounts are considered in the overall percentages of silicone oil present in the compositions of the present invention.

Suitable for use herein are silicone elastomers which can be emulsifying or non-emulsifying crosslinked siloxane elastomers or mixtures thereof. No specific restriction exists as to the type of curable organopolysiloxane composition which can serve as starting material for the crosslinked organopolysiloxane elastomer.

The compositions of the present invention may include an emulsifying crosslinked organopolysiloxane elastomer, a non-emulsifying crosslinked organopolysiloxane elastomer, or a mixture thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Non-emulsifying elastomers useful in the present invention are formed via crosslinking organohydroenpolysiloxanes with an alpha, omega-diene. Emulsifying elastomers herein include polyoxyalkylene modified elastomers formed via crosslinking from organohydrogenpolysiloxanes with polyoxyalkylene dienes or organohydrogenpolysiloxanes containing at least one polyether group crosslinked with an alpha, omega-diene. Emulsifying crosslinked organopolysiloxane elastomer can notably be chosen from the crosslinked polymers described in U.S. Pat. Nos. 5,412,004 (issued May 2, 1995); 5,837,793 (issued Nov. 17, 1998); and 5,811,487 (issued Sep. 22, 1998). In addition, an emulsifying elastomer comprised of dimethicone copolyol crosspolymer (and dimethicone) is available from Shin Etsu under the tradename KSG-21.

Non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. No. 4,970,252 to Sakuta, et al., issued Nov. 13, 1990; U.S. Pat. No. 5,760,116 to Kilgour, et al., issued Jun. 2, 1998; U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK. Commercially available elastomers preferred for use herein are Dow Corning's 9040 silicone elastomer blend, Shin Etsu's KSG-21, and mixtures thereof

b) Mineral Oil

Mineral oil, which is also known as petrolatum liquid, is a mixture of liquid hydrocarbons obtained from petroleum. See The Merck Index, Tenth Edition, Entry 7048, p. 1033 (1983) and International Cosmetic Ingredient Dictionary, Fifth Edition, vol. 1, p.415-417 (1993).

c) Petrolatum

Petrolatum, which is also known as petroleum jelly, is a colloidal system of nonstraight-chain solid hydrocarbons and high-boiling liquid hydrocarbons, in which most of the liquid hydrocarbons are held inside the micelles. See The Merck Index, Tenth Edition, Entry 7047, p. 1033 (1983); Schindler, Drug. Cosmet. Ind., 89, 36-37, 76, 78-80, 82 (1961); and International Cosmetic Ingredient Dictionary, Fifth Edition, vol. 1, p. 537 (1993).

d) Straight and Branched Chain Hydrocarbons Having from About 7 to About 40 Carbon Atoms

Nonlimiting examples of these hydrocarbon materials include dodecane, isododecane, squalane, cholesterol, hydrogenated polyisobutylene, docosane (i.e. a C₂₂ hydrocarbon), hexadecane, isohexadecane (a commercially available hydrocarbon sold as Permethyl.RTM. 101A by Presperse, South Plainfield, N.J.). Also useful are the C₇ -C₄₀ isoparaffins, which are C₇ -C₄₀ branched hydrocarbons.

e) C₁-C₃₀ Alcohol Esters of C₁-C₃₀ Carboxylic Acids and of C₂- C₃₀ Dicarboxylic Acids Including Straight and Branched Chain Materials as well as Aromatic Derivatives (As Used Herein in Reference to the Hydrophobic Component, Mono- and Poly-carboxylic Acids Include Straight Chain, Branched Chain and Aryl Carboxylic Acids)

Nonlimiting examples include diisopropyl sebacate, diisopropyl adipate, isopropyl myristate, isopropyl palmitate, methyl palmitate, myristyl propionate, 2-ethylhexyl palmitate, isodecyl neopentanoate, di-2-ethylhexyl maleate, cetyl palmitate, myristyl myristate, stearyl stearate, isopropyl isostearate, methyl stearate, cetyl stearate, behenyl behenrate, dioctyl maleate, dioctyl sebacate, diisopropyl adipate, cetyl octanoate, and diisopropyl dilinoleate.

f) Mono-, Di- and Tri-glycerides of C₁-C₃₀ Carboxylic Acids

Non-limiting examples of such thickening agents include caprylic/capric triglyceride, PEG6 caprylic/capric triglyceride, PEG-8 caprylic/capric triglyceride, etc.

g) Alkylene Glycol Esters of C₁-C₃₀ Carboxylic Acids

Suitable thickening agents include ethylene glycol mono- and di-esters, and propylene glycol mono- and di-esters of C₁, -C₃₀ carboxylic acids (e.g., ethylene glycol distearate).

h) Propoxylated and Ethoxylated Derivatives of the Foregoing Materials

i) C₁-C₃₀ Mono- and Poly-esters of Sugars and Related Materials

These esters are derived from a sugar or polyol moiety and one or more carboxylic acid moieties. Depending on the constituent acid and sugar, these esters can be in either liquid or solid form at room temperature. Examples of liquid esters include: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. A preferred solid material is sucrose polyester in which the degree of esterification is 7-8, and in which the fatty acid moieties are C₁₈ mono- and/or di-unsaturated and behenic, in a molar ratio of unsaturates:behenic of 1:7 to 3:5. A particularly preferred solid sugar polyester is the octaester of sucrose in which there are about 7 behenic fatty acid moieties and about 1 oleic acid moiety in the molecule. Other materials include cottonseed oil or soybean oil fatty acid esters of sucrose.

j) Organopolysiloxane Oils

In preferred embodiments, the hydrophobic phase is a silicone oil phase and the continuous silicone phase contains an organopolysiloxane oil. Such organopolysiloxane oil may be volatile, non-volatile, or a mixture of volatile and non-volatile silicones. The term “nonvolatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Examples of suitable organopolysiloxane oils include polyalkylsiloxanes, cyclic polyalkylsiloxanes, and polyalkylarylsiloxanes.

Polyalkylsiloxanes useful in the composition herein include polyalkylsiloxanes with viscosities of from about 0.5 to about 1,000,000 centistokes at 25° C. Such polyalkylsiloxanes can be represented by the general chemical formula R₃SiO[R₂SiO]ₓSiR₃ wherein R is an alkyl group having from one to about 30 carbon atoms (preferably R is methyl or ethyl, more preferably methyl; also mixed alkyl groups can be used in the same molecule), and x is an integer from 0 to about 10,000, chosen to achieve the desired molecular weight which can range to over about 10,000,000. Commercially available polyalkylsiloxanes include the polydimethylsiloxanes, which are also known as dimethicones, examples of which include the Vicasil® series sold by General Electric Company and the Dow Corning® 200 series sold by Dow Corning Corporation. Specific examples of suitable polydimethylsiloxanes include Dow Corning® 200 fluid having a viscosity of 0.65 centistokes and a boiling point of 100° C, Dow Corning® 225 fluid having a viscosity of 10 centistokes and a boiling point greater than 200° C, and Dow Corning® 200 fluids having viscosities of 50, 350, and 12,500 centistokes, respectively, and boiling points greater than 200° C. Suitable dimethicones include those represented by the chemical formula (CH₃)₃SiO[(CH₃)₂SiO]ₓ[CH₃RSiO]_{y}Si(CH₃)₃ wherein R is straight or branched chain alkyl having from two to about 30 carbon atoms and x and y are each integers of 1 or greater selected to achieve the desired molecular weight which can range to over about 10,000,000. Examples of these alkyl-substituted dimethicones include cetyl dimethicone and lauryl dimethicone.

Cyclic polyalkylsiloxanes suitable for use in the composition include those represented by the chemical formula [SiR₂—O]ₙ wherein R is an alkyl group (preferably R is methyl or ethyl, more preferably methyl) and n is an integer from about 3 to about 8, more preferably n is an integer from about 3 to about 7, and most preferably n is an integer from about 4 to about 6. When R is methyl, these materials are typically referred to as cyclomethicones. Commercially available cyclomethicones include Dow Corning® 244 fluid having a viscosity of 2.5 centistokes, and a boiling point of 172° C., which primarily contains the cyclomethicone tetramer (i.e. n=4), Dow Corning® 344 fluid having a viscosity of 2.5 centistokes and a boiling point of 178° C., which primarily contains the cyclomethicone pentamer (i.e. n=5), Dow Corning® 245 fluid having a viscosity of 4.2 centistokes and a boiling point of 205° C., which primarily contains a mixture of the cyclomethicone tetramer and pentamer (i.e. n=4 and 5), and Dow Corning® 345 fluid having a viscosity of 4.5 centistokes and a boiling point of 217°, which primarily contains a mixture of the cyclomethicone tetramer, pentamer, and hexamer (i.e. n=4, 5, and 6).

Also useful are materials such as trimethylsiloxysilicate, which is a polymeric material corresponding to the general chemical formula [(CH₂)₃SiO_{½}]ₓ[SiO₂]Y, wherein x is an integer from about 1 to about 500 and y is an integer from about 1 to about 500. A commercially available trimethylsiloxysilicate is sold as a mixture with dimethicone as Dow Corning® 593 fluid.

Dimethiconols are also suitable for use in the composition. These compounds can be represented by the chemical formulas R₃SiO[R₂SiO]ₓSiR₂OH and HOR₂SiO[R₂SiO]ₓSiR₂OH wherein R is an alkyl group (preferably R is methyl or ethyl, more preferably methyl) and x is an integer from 0 to about 500, chosen to achieve the desired molecular weight. Commercially available dimethiconols are typically sold as mixtures with dimethicone or cyclomethicone (e.g. Dow Corning® 1401, 1402, and 1403 fluids).

Polyalkylaryl siloxanes are also suitable for use in the composition. Polymethylphenyl siloxanes having viscosities from about 15 to about 65 centistokes at 25° C. are especially useful.

Preferred for use herein are organopolysiloxanes selected from the group consisting of polyalkylsiloxanes, alkyl substituted dimethicones, cyclomethicones, trimethylsiloxysilicates, dimethiconols, polyalkylaryl siloxanes, and mixtures thereof. More preferred for use herein are polyalkylsiloxanes and cyclomethicones. Preferred among the polyalkylsiloxanes are dimethicones.

A continuous silicone phase may contain one or more non-silicone oils. However, in preferred embodiments, the concentrations of non-silicone oils in the continuous silicone phase are minimized or avoided altogether so as to further enhance the delivery of the oil-soluble skin care active. Suitable non-silicone oils for use in combination with silicone oils have a melting point of about 25° C. or less under about one atmosphere of pressure.

k) Vegetable Oils and Hydrogenated Vegetable Oils

Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, and mixtures thereof.

l) Animal Fats and Oils

Animal fats and oils include, for example, lanolin and derivatives thereof, and cod liver oil.

m) Also useful are C4-C20 alkyl ethers of polypropylene glycols, C1-C20 carboxylic acid esters of polypropylene glycols, and di-C8-C30 alkyl ethers. Nonlimiting examples of these materials include PPG-14 butyl ether, PPG-15 stearyl ether, dioctyl ether, dodecyl octyl ether, and mixtures thereof.

2. Hydrophilic Phase

Emulsions of the present invention also comprise a hydrophilic phase which includes water and/or other hydrophilic diluents. Preferred emulsions contain a dermatologically acceptable, hydrophilic diluent. As used herein, “diluent” includes materials in which the other optional components can be dispersed, dissolved, or otherwise incorporated. Nonlimiting examples of hydrophilic diluents are water, organic hydrophilic diluents such as lower monovalent alcohols (e.g., C1-C4) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, butanediol, ether propanol, ethoxylated ethers, propoxylated ethers and combinations thereof. Water is a preferred diluent.

The composition preferably comprises from about 10% to about 75%, by weight of the composition formed, of a hydrophilic phase. The hydrophilic phase can thus comprise water, or a combination of water and one or more water soluble or dispersible ingredients. Hydrophilic phases comprising at least 30% of water, by weight of the phase, are preferred.

3. Emulsifiers

The water-in-oil emulsions of the present invention preferably comprise an emulsifier for dispersing the aqueous phase. In a preferred embodiment, the composition contains from about 0.1% to about 10% emulsifier, more preferably from about 0.5% to about 7.5%, most preferably from about 1% to about 5%, by weight of the composition formed, of an emulsifier. The emulsifier helps disperse and suspend the aqueous phase within the continuous oil phase.

A wide variety of emulsifying agents can be employed herein to form the water-in-oil emulsion. Known or conventional emulsifying agents can be used in the composition, provided that the selected emulsifying agent is chemically and physically compatible with essential components of the composition, and provides the desired dispersion characteristics. Suitable emulsifiers include silicone emulsifiers, non-silicon-containing emulsifiers, and mixtures thereof, known by those skilled in the art for use in topical personal care products. Preferably these emulsifiers have an HLB value of less than about 6. Emulsifiers having an HLB value greater than 6 can be used in combination with other emulsifiers to achieve an effective weighted average HLB for the combination that falls within these ranges.

Emulsifying silicone elastomers are preferred for use herein and are discussed more fully above. Other silicone emulsifiers are also preferred. A combination of emulsifying silicone elastomer and silicone emulsifier is also useful herein.

A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

The dimethicone copolyol emulsifiers useful herein can be described by the following general structure:

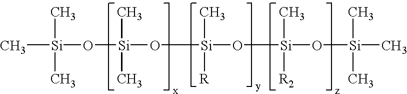

wherein R is C1-C30 straight, branched, or cyclic alkyl and R² is selected from the group consisting of

—(CH₂)ₙ—O—(CH₂CHR³O)ₘ—H

and

—(CH₂)ₙ—O—(CH₂CHR³O)ₘ—(CH₂CHR⁴O)ₒ—H

wherein n is an integer from 3 to about 10; R³ and R⁴ are selected from the group consisting of H and C1-C6 straight or branched chain alkyl such that R³ and R⁴ are not simultaneously the same; and m, o, x, and y are selected such that the molecule has an overall molecular weight from about 200 to about 10,000,000, with m, o, x, and y being independently selected from integers of zero or greater such that m and o are not both simultaneously zero, and z being independently selected from integers of 1 or greater. It is recognized that positional isomers of these copolyols can be achieved. The chemical representations depicted above for the R² moieties containing the R³ and R⁴ groups are not meant to be limiting but are shown as such for convenience.

Also useful herein, although not strictly classified as dimethicone copolyols, are silicone surfactants as depicted in the structures in the previous paragraph wherein R² is:

—(CH₂)ₙ—O—R⁵

wherein R⁵ is a cationic, anionic, amphoteric, or zwitterionic moiety.

Nonlimiting examples of dimethicone copolyols and other silicone surfactants useful as emulsifiers herein include polydimethylsiloxane polyether copolymers with pendant polyethylene oxide sidechains, polydimethylsiloxane polyether copolymers with pendant polypropylene oxide sidechains, polydimethylsiloxane polyether copolymers with pendant mixed polyethylene oxide and polypropylene oxide sidechains, polydimethylsiloxane polyether copolymers with pendant mixed poly(ethylene)(propylene)oxide sidechains, polydimethylsiloxane polyether copolymers with pendant organobetaine sidechains, polydimethylsiloxane polyether copolymers with pendant carboxylate sidechains, polydimethylsiloxane polyether copolymers with pendant quaternary ammonium sidechains; and also further modifications of the preceding copolymers containing pendant C2-C30 straight, branched, or cyclic alkyl moieties. Examples of commercially available dimethicone copolyols useful herein sold by Dow Coming Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples of dimethicone copolyols also include lauryl dimethicone copolyol, dimethicone copolyol acetate, diemethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate. See International Cosmetic Ingredient Dictionary, Fifth Edition, 1993.

Non-limiting examples of non-silicone-containing emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

Nonlimiting examples of suitable non-silicone-containing emulsifiers for use herein include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

II. Dispenser

The skin care kit of the present invention comprises a dispensing container, such as a pump dispenser for the above described skin care composition. Non-limiting examples of pump dispensers useful herein include dip tube pumps and positive displacement pumps. The dispensing containers are capable of dispensing a predetermined amount for use by consumers to spread a quantity of product over a large surface area of the skin. These dispensing containers are necessary in order to distribute an adequate amount of the composition so as to provide the enhanced skin care benefits previously described.

Dip tube pumps can include the side actuator “Aquarius” pump available from available from RPC-Bramlage-Wiko, located in Pulheim, Germany. Among the positive displacement pumps available is the “Magic C” pump also available from RPC-Bramlage-Wiko . In both cases, the product dispenses up through the dispensing or top surface of the container. In the case of Magic C, an upper valve serves as a seal at the same level as the dispensing or top surface of the actuator.

The dispenser comprises a container for storing a supply of the skin care composition to be dispensed, said container containing from about 1 oz or 30 ml to about 4 oz or 120 ml. The dimensions of the container are preferably from about 4 cm by about 2.5 cm to about 20 cm × about 7 cm. Preferred dimensions of the container are 9.0 cm by 4.5 cm. (Height×width)

This dispenser may also comprise a manually operated pump which is fixedly connected to a container having an actuator cap. As used herein, “fixedly” means that the pump is not easily removed from the container without destroying the dispenser.

The container can be formed in a wide variety of shapes which include, but are not limited to, substantially cylindrical, oval, elliptical, rectangular, triangular, and combinations thereof. The cylindrical embodiment is shown in the figures contained herein.

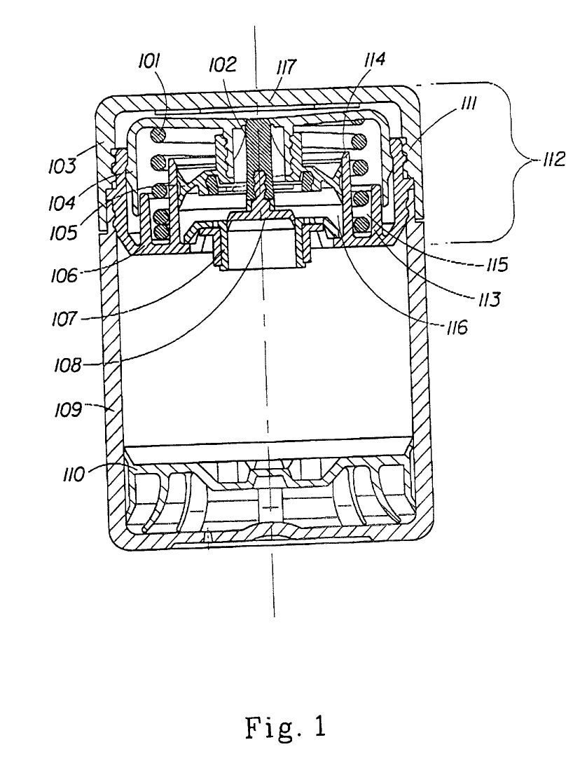

Depicted in FIG. 1 is a first preferred embodiment of the invention showing the main components of a dispenser for the present skin care compositions which include a container 109, a headpiece 112 extending therefrom, a closure cap 103 for covering the actuation surface 104, and a follower piston 110 slidably mounted for displacement within container 109. The major components of the dispenser are made of an injection-moldable plastic, preferably polyethylene, polypropylene, or polyethylene terepthalate, so that dispenser is of a lightweight construction, and the present skin care composition which is filled into container 109 of the dispenser is unaffected by the material of the dispenser. The skin care composition is advanced within container 109 by the displacement of follower piston 110 along the interior wall surface of container 109 by the action thereon of the surrounding atmospheric pressure, so that the follower piston 110 rises within container 109 during each use. In this manner, as the quantity of the skin care composition within container 109 is reduced with each use, the follower piston 110 raises the product to keep it in contact with the dispensing mechanism incorporated in headpiece 112. Retention ring 106 of headpiece 112 is offset radially inwards of the peripheral wall of container 109 to thereby form a seat for screw cap 103, permitting it to be seated on container 109 in alignment with its peripheral wall surface, so that the dispenser as a whole has a smooth outer shape.

The retention ring 106 is formed with an upper wall including screw threads 111 which engage the thread inside the outer wall of screw cap 103. Furthermore, the retention ring 106 and screw cap 103 are designed such that the screw cap 103 and container 109 are united with their peripheral wall surfaces in alignment without a gap there between.

Integrally formed with retention ring 106 and extending axially therefrom is an outer sleeve 113 and an inner sleeve 114. The outer diameter of outer sleeve 113 is smaller than that of actuator surface 104 to provide sufficient clearance for the downward motion of the actuation surface 104 during consumer use. An annular space 115, defined within outer sleeve 113 by an inner sleeve 114, serves as a locating mechanism for pressure spring 101. Located coaxially within inner sleeve 114 is valve ring 107. This valve ring 107 is fits within an opening in the lower wall of retention ring 106 and functions as a non-return valve. Seated within the retention ring 106, the valve ring 107 preventing product flow backwards within the dispenser system (from the pump chamber 116 into the container 109) while permitting flow in the opposite direction (from the container 109 into the pump chamber 116).

Valve piston 108 is in sealingly and slidable engagement with the inner diameter opening of valve ring 107. Fixedly attached to, or conceivably integral to, the valve piston 108 is the valve pin 102. The valve piston 108/valve pin 102 assembly operates as a unit to control the upper valve/dispensing orifice 117 at the dispensing surface of the actuation surface 104. As the user depresses the actuation surface 104, the pressure piston 105 reduces the volume of the pump chamber 116 building internal pressure within the pump chamber 116. This internal pressure forces the valve piston 108/valve pin 102 assembly downwards, opening the upper valve/dispensing orifice 117 allowing product to escape the pump chamber and flow through the upper valve/dispensing orifice 117, typically but not necessarily at the center of actuation surface 104.

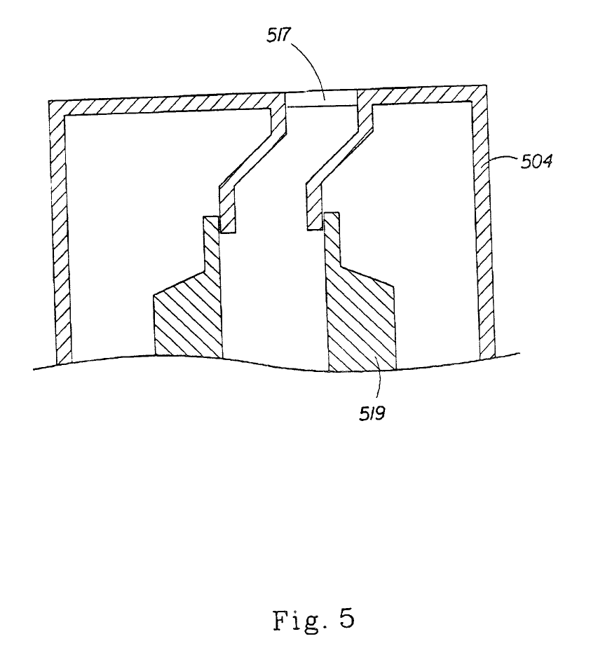

Integral to valve pin 102 is a flexible structure providing a bias to keep the valve pin 102/valve piston 108 assembly in an upward resting position, thus sealing the upper valve/dispensing orifice 117. The integral flexible structure of valve pin 102 includes an outer ring which is fixedly assembled to pressure piston 105 in a coaxial fashion. Pressure piston 105 is fixedly connected to actuation surface 104 with a snap fit such that both are slidably engaged with retention ring 106. Actuation surface 104 is of a generally cup-shaped configuration comprising a top wall and an annular outer wall. Within the space defined by top wall and outer wall, actuation surface 104 is provided with a tubular section axially extending from the top wall downward fixedly engaging with the pressure piston 105. In an alternative embodiment as shown in FIG. 5, the tubular section extends angularly downward from the dispensing orifice 517 located off-center of the actuation surface 504.

The sealingly slidable engagement of pressure piston 105 with the interior wall surface of inner sleeve 114 results in the formation of a pump chamber 116 between a bottom portion of pressure piston 105 and valve ring 107 and valve piston 108, the volume of pump chamber 116 being variable in response to axial displacement of actuation surface 104 and thus pressure piston 105. The bottom portion of pressure piston 105 is formed with an opening allowing product passage up to the upper valve/dispensing orifice 117.

Disposed in the annular space between inner sleeve 114 and outer sleeve 113 is pressure spring 101 acting as a return spring for actuation surface 104 and held under compression between the bottom wall of retention ring 106 and the top wall of actuation surface 104, so that in the absence of an actuating force actuation surface 104 is maintained in the upward position shown in FIG. 1.

The top surface of actuation surface 104 forms an actuating surface for the application of an axially downwards directed actuating force for dispensing the skin care composition from the dispenser 101 through a dispensing orifice 117.

The above described dispenser operates as follows: On the first actuation of actuation surface 104, it may be assumed that only container 109 is filled with the skin care composition, so that axial depression of actuation surface 104 initially results in a “dead” stroke of pressure piston 105 to reduce the volume of pump chamber 116. The resultant pressure rise in pump chamber 116 causes the valve piston 108/valve pin 102 assembly to move downwards, thus opening the upper valve 117 to permit the air to escape from pump chamber 116 through the dispensing orifice 117. On subsequent release of the actuating force acting on actuation surface 104, pressure spring 101 acts to return actuation surface 104 upwards to its starting position, whereby the volume of pump chamber 116 is again increased. The resultant vacuum within pump chamber 116 and the spring force of the deflection in the flexible support structure of valve pin 102 causes the valve pin 102 and valve piston 108 to return to their rest position obturating dispensing orifice 117. The same vacuum pressure within pump chamber 116 forces the outer sealing ring of the valve ring 107 to be lifted off the mating surface of retention ring 106, opening a passage to thereby permit the skin care composition to flow from container 109 into pump chamber 116 until a pressure equilibrium is established between pump chamber 116 and the interior of container 109, whereupon the sealing ring of valve ring 107 may close again. Renewed depression of actuation surface 104 on the one hand causes the pressure acting on valve ring 107 to be increased to thereby completely interrupt communication between pump chamber 116 and the interior of container 109, and on the other hand causes valve piston 108 and valve ring 102 to be pushed downward, so that the skin care composition is expelled through the upper valve/dispensing orifice 117.

The amount of the skin care composition dispensed is thus determined by the length of the piston stroke expelling the product from pump chamber 116 through upper valve/dispensing orifice 117. When the pressure acting on actuation surface 104 is again relieved, pressure spring 101 again acts to return actuation surface 104 to its rest position, the resultant vacuum in pump chamber 116 causing valve piston 108 and valve ring 102 to move upwards to their rest position, closing the upper valve/dispensing orifice 117. At the same time, the vacuum generated in pump chamber 116 causes sealing ring of the valve ring 108 between the product supply and pump chamber 116 to be opened, so that the skin care composition flows from the interior of container 109 into pump chamber 116 until the latter is again filled with the product and the sealing ring of the valve ring 108 is permitted to return to its closure position on the bottom wall of the retention ring 106 by the pressure equilibrium thus established.

It is of course also possible to likewise fill pump chamber 116 with the skin care composition prior to the first actuation of the dispenser, so that the first depression of actuation surface 104 results in the skin care composition to be dispensed from the dispenser.

Furthermore, the dispenser alternatively comprises an manually-operated pump fixedly connected to an ergonomic container having an actuator cap such that the dispenser is configured so that the pump is in register with the container and the container is shaped so as to provide for comfortable and easy gripping by a human hand. The hand should readily conform to the shape of the container and the actuator can be depressed substantially solely by movement of the tip of either the thumb or index finger.

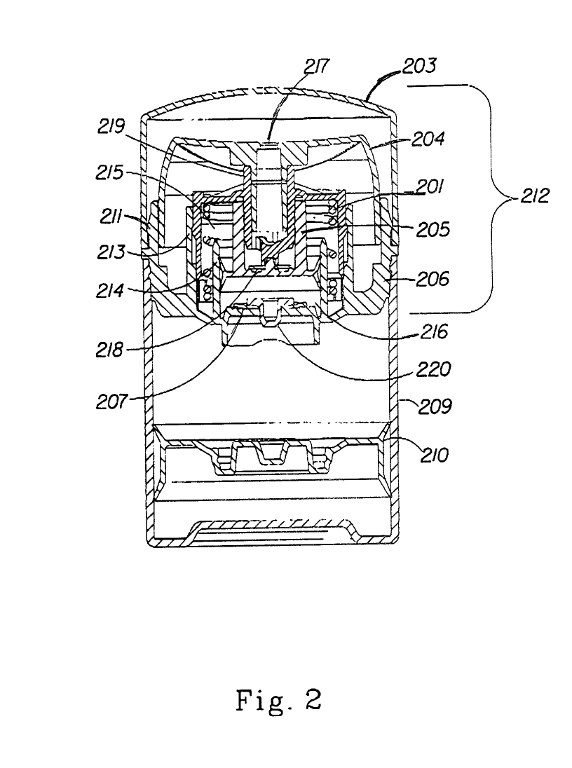

Depicted in FIG. 2 is a second embodiment of the invention showing the main components of a dispenser for the present skin care compositions which include a container 209, a headpiece 212 extending therefrom, a closure cap 203 for covering the actuator surface 204, and a follower piston 210 slidably mounted for displacement within container 209. The major components of the dispenser are made of an injection-moldable plastic, preferably polyethylene, polypropylene, or polyethylene terepthalate, so that the dispenser of FIG. 2 is of a lightweight construction, and the present skin care composition which is filled into container 209 of the dispenser is unaffected by the material of the dispenser. The skin care composition is advanced within container 209 by the displacement of follower piston 210 along the interior wall surface of container 209 by the action thereon of the surrounding atmospheric pressure, so that the follower piston 210 rises within container 209 during each use. In this manner, as the quantity of the skin care composition within container 209 is reduced with each use, the follower piston 210 raises the product to keep it in contact with the dispensing mechanism incorporated in headpiece 212.

Retention ring 206 of headpiece 212 is flush radially to the peripheral wall of container 209 to thereby form a seat for cap 202, permitting it to be seated on retention ring 206 in alignment with its peripheral wall surface, so that the dispenser as a whole has a smooth outer shape.

The retention ring 206 is formed with an upper wall including snap bead 211 which engages the inner snap bead of cap 203. Furthermore, the retention ring 206 and cap 203 are designed such that the cap 203, retention ring 206, and container 209 are united with their peripheral wall surfaces in alignment without a gap there between.

Integrally formed with retention ring 206, and extending axially there from, is an outer sleeve 213 and an inner sleeve 214. The outer diameter of outer sleeve 213 is smaller than that of actuator surface 204 to provide sufficient clearance for the downward motion of the actuator surface during consumer use. An annular space 215, defined within outer sleeve 213 by an inner sleeve 214, and a spring housing 219, serves as a locating mechanism for pressure spring 201. Located coaxially within inner sleeve 214 is valve 207. This valve 207 is fixed to the lower wall of retention ring 206 by valve plug 220 and functions as a non-return valve. Valve 207 prevents product flow backwards within the dispenser system (from the pump chamber 216 into the container 209) while permitting flow in the opposite direction (from the container 209 into the pump chamber 216).

Pressure piston 205 is in sealingly slidable engagement with the inner diameter of sleeve 214. Fixedly attached to, or conceivably integral to, the pressure piston 205 is the spring housing 219 which is slidably engaged to sleeve 213. The pressure piston 205/spring housing 219 assembly operates as a unit to control the upper valve 218. The upper valve 218 is fixed to the upper wall of pressure piston 205 by a vertical projection integral to, or separate from, the spring housing 219. Actuation surface 204 is fixedly attached to, or conceivable integral to, the spring housing 219 such that both are slidably engaged with retention ring 206, and provides a tubular pathway for product to flow from the upper valve 218 to the orifice 217.

As the user depresses the actuation surface 204, the pressure piston 205 reduces the volume of the pump chamber 216 building internal pressure within the pump chamber 216. This internal pressure forces the pressure piston 205/spring housing 219 assembly downwards, opening the upper valve 218 allowing product to escape the pump chamber and flow through the upper valve 218 to the orifice 217, typically but not necessarily at the center of actuation surface 204. Actuation surface 204 is of a generally cup-shaped configuration comprising a top wall and an annular outer wall. Within the space defined by top wall and outer wall, actuation surface 204 is provided with a tubular section axially extending from the top wall downward fixedly engaging with the spring housing 219. Said actuation surface 204 can have a round planar circumstantial shape, but does not exclude other shapes, such as an elliptical planar shape wherein the retention ring 206 and spring housing 219 are such to accommodate the selected actuator surface 204 shape. An alternative embodiment of said actuation surface 204 is one having a saddle shape, that is a shallow, evenly tapered “U” shaped recess traversing the diameter of said actuation surface 204.

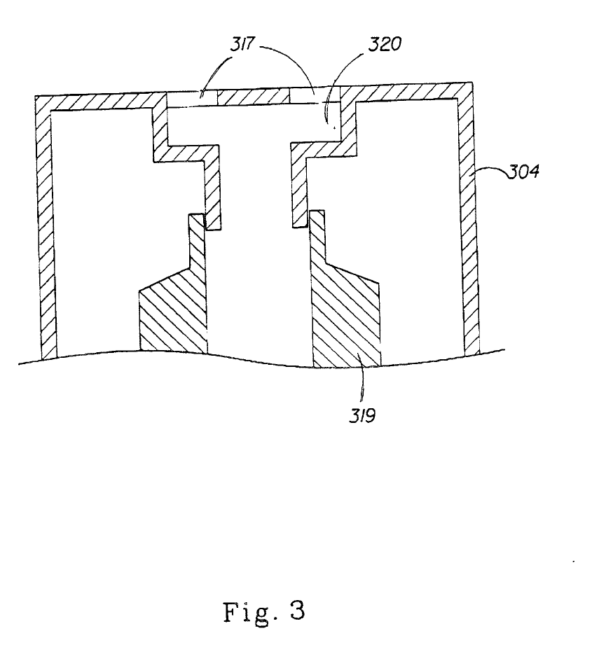

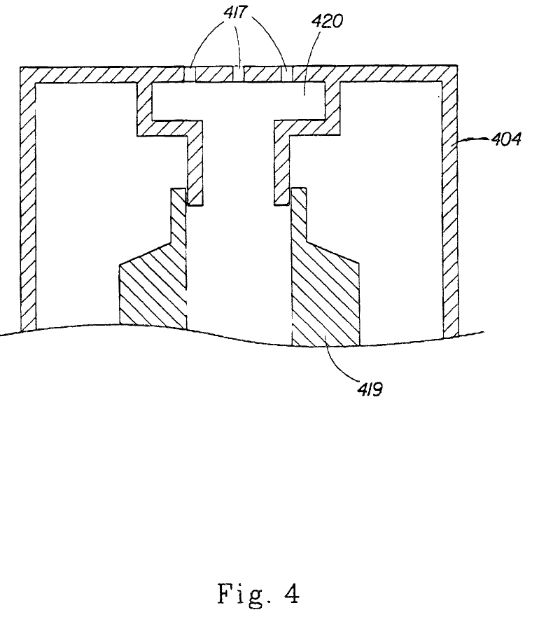

Outer sleeve 213 cooperates with inner sleeve 214 to form guide and retention means for the actuation surface 204 and pressure piston 205 simultaneously acting as the dispensing mechanism of the dispenser. The sealingly slidable engagement of pressure piston 205 with the interior wall surface of inner sleeve 214 results in the formation of a pump chamber 216 between a bottom portion of pressure piston 205 and valve 207 and retention ring 206, the volume of pump chamber 216 being variable in response to axial displacement of actuation surface 204 and thus pressure piston 205. The bottom portion of pressure piston 205 is formed with an opening allowing product passage up to the upper valve 218, through the tubular section and out dispensing orifice 217. In an alternative embodiment as shown in FIG. 5, the tubular section extends angularly downward from the dispensing orifice 517 located off-center of the actuation surface 504. Alternative embodiments in FIGS. 3 and 4 provide multiple dispensing orifices 317 and 417 respectively. FIG. 3 shows wherein actuation surface 304 provides for product to be dispensed through a reservoir 320 through a plurality of dispensing orifices 317 during dosing of the composition. FIG. 3 shows a simplified version of separately molded pathways within the actuator structure connected at a common point above or within passage from spring housing 319. These pathways can be integral with the top surface only, or could extend from the actuation surface 304 to the connection point. FIG. 4 shows an assembly having a separate reservoir 420 for collecting product as it exits the passage from spring housing 419, allowing for expulsion of product as pressure builds through multiple dispensing orifices 417 contained within the “reservoir” geometry.

Disposed in the annular space between inner sleeve 214 and outer sleeve 213 is pressure spring 201 acting as a return spring for actuation surface 204 and held under compression between the bottom wall of retention ring 206 and the bottom wall of spring housing 219, so that in the absence of an actuating force, actuation surface 204 is maintained in the upward position shown in FIG. 2.

The top surface of actuation surface 204 forms the actuator that upon application of an axially downward force, results in an actuating force for dispensing the skin care composition from dispenser 202 through a dispensing orifice 217.

The above described dispenser of FIG. 2 operates as follows: On the first actuation of dispenser, it may be assumed that only container 209 is filled with the skin care composition, so that axial depression of actuation surface 204 initially results in a “dead” stroke of pressure piston 205 to reduce the volume of pump chamber 216. The resultant pressure rise in pump chamber 216 causes upper valve 218 to open permitting the air to escape from pump chamber 216 through the dispensing orifice 217. On subsequent release of the actuating force acting on the actuation surface 204, pressure spring 201 acts to return actuation surface 204 upwards to its starting position, whereby the volume of pump chamber 216 is again increased. It is of course also possible to likewise fill pump chamber 216 with the skin care composition prior to the first actuation of dispenser 202, so that the first depression of actuation surface 204 results in the skin care composition to be dispensed from the dispenser.

The resultant vacuum formed within pump chamber 216 after depressing the actuator surface causes upper valve 218 to close against the top of pressure piston 205. The same vacuum pressure within pump chamber 216 forces valve 207 to be lifted off the mating surface of retention ring 206, opening a passage to thereby permit the skin care composition to flow from container 209 into pump chamber 216 until actuation surface 204 reaches its most upward position, whereupon valve 207 may close again.

When the pressure acting on actuation surface 204 is again relieved, pressure spring 201 again acts to return said actuation surface 204 to its rest position, the resultant vacuum in said pump changer 216 closing the valve 218. At the same time, the vacuum generated in said pump chamber 216 causes valve 217, between the product supply and pump chamber 216, to be opened, allowing the skin care composition to flow from the interior of said 209 into pump chamber 216 until the latter is again filled with the product

While the actual amount of the skin care composition dispensed is thus determined by the user's extent of actuating the actuation surface 204, the maximum amount of the composition expelled from the package depends on the length of the piston stroke of the pump. In the package of the present invention the maximum amount of the composition expelled by full depressing the actuation surface 204 wherein the product from pump chamber 216 through dispensing orifice 217 is from about 0.75 ml to about 1.25 ml. When the pressure acting on actuation surface 204 is again relieved, pressure spring 201 again acts to return actuation surface 204 to its rest position, the resultant vacuum in pump chamber 216 closing the upper valve 218. At the same time, the vacuum generated in pump chamber 216 causes valve 207 between the product supply and pump chamber 216 to be opened, so that the skin care composition flows from the interior of container

Optional Components

The compositions of the present invention may contain one or more optional components. Preferred compositions for use herein include one or more skin care actives. Such skin care actives may be included as a substantially pure material, or as an extract obtained by suitable physical and/or chemical isolation from natural (e.g., plant) sources.

In a preferred embodiment, where the composition is to be in contact with human keratinous tissue, the additional components(s) should be suitable for application to keratinous tissue, that is, when incorporated into the composition they are suitable for use in contact with human keratinous tissue without undue toxicity, incompatibility, instability, allergic response, and the like within the scope of sound medical judgment. The CTFA Cosmetic Ingredient Handbook, Second Edition (1992) describes a wide variety of cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucoside ascorbyl glucosamine, pyridoxine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents (e.g. vitamin D compounds, mono-, di-, and tri-terpenoids, beta-ionol, cedrol), thickeners, and vitamins and derivatives thereof.

In any embodiment of the present invention, however, the components useful herein can be categorized by the benefit they provide or by their postulated mode of action. However, it is to be understood that the components useful herein can in some instances provide more than one benefit or operate via more than one mode of action. Therefore, classifications herein are made for the sake of convenience and are not intended to limit the component to that particular application or applications listed. available,

Phytosterols

Phytosterol and derivatives thereof are known for providing skin lightening benefits. Non-limiting examples of oil-soluble phytosterol derivatives include β-sitosterol, campesterol, brassicasterol, lupenol, α-spinasterol, stigmasterol, their derivatives, and combinations thereof. More preferably, the phytosterol derivative is selected from the group consisting of β-sitosterol, campesterol, brassicasterol, stigmasterol, their derivatives, and combinations thereof.

Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company (Milwaukee, Wis.), Sigma Chemical Company (St. Louis, Mo.), and Dragoco (Totowa, N.J.).

Desquamation Actives

A safe and effective amount of a desquamation active may be added to the compositions of the present invention, preferably from about 0.1% to about 10%, more preferably from about 0.2% to about 5%, even more preferably from about 0.5% to about 4%, by weight of the composition. Desquamation actives enhance the skin appearance benefits of the present invention. For example, the desquamation actives tend to improve the texture of the skin (e.g., smoothness). One desquamation system that is suitable for use herein contains sulfhydryl compounds and zwitterionic surfactants and is described in U.S. Pat. No. 5,681,852, to Bissett, incorporated herein by reference. Another desquamation system that is suitable for use herein contains salicylic acid and zwitterionic surfactants and is described in U.S. Pat. No. 5,652,228 to Bissett, incorporated herein by reference. Zwitterionic surfactants such as described in these applications are also useful as desquamatory agents herein, with cetyl betaine being particularly preferred.

Anti-Acne Actives

The compositions of the present invention may contain a safe and effective amount of one or more anti-acne actives preferably from about 0.01% to about 50%, more preferably from about 1% to about 20%. Examples of useful anti-acne actives include resorcinol, sulfur, salicylic acid, benzoyl peroxide, erythromycin, zinc, etc. Further examples of suitable anti-acne actives are described in further detail in U.S. Pat. No. 5,607,980, issued to McAtee et al, on Mar. 4, 1997.

Anti-Wrinkle Actives/Anti-Atrophy Actives

The compositions of the present invention may contain a safe and effective amount of one or more anti-wrinkle actives or anti-atrophy actives. Exemplary anti-wrinkle/anti-atrophy actives suitable for use in the compositions of the present invention include hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, skin peel agents (e.g., phenol and the like), vitamin B₃ compounds and retinoids which enhance the keratinous tissue appearance benefits of the present invention, especially in regulating keratinous tissue condition, e.g., skin condition.

a) Vitamin B₃ Compounds

The compositions of the present invention may contain a safe and effective amount of a vitamin B₃ compound. When vitamin B₃ compounds are present in the compositions of the instant invention, the compositions preferably contain from about 0.01% to about 50%, more preferably from about 0.1% to about 10%, still more preferably from about 1% to about 5%, and still more preferably from about 2% to about 5%, by weight of the composition, of the vitamin B₃ compound.

As used herein, “vitamin B₃ compound” means a compound having the formula:

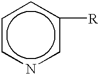

wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B₃ compounds include nicotinic acid esters, including non-vasodilating esters of nicotinic acid (e.g., tocopheryl nicotinate and niacinamide), nicotinyl amino acids, nicotinyl alcohol esters of carboxylic acids, nicotinic acid N-oxide and niacinamide N-oxide.

b) Retinoids

The compositions of the present invention may contain a safe and effective amount of a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C22 alkyl esters of retinol, including retinyl palmitate, retinyl acetate, retinyl propionate), retinal, and/or retinoic acid (including all-trans retinoic acid and/or 13-cis-retinoic acid), or mixtures thereof. More preferably the retinoid is a retinoid other than retinoic acid. These compounds are well known in the art and are commercially available from a number of sources, e.g., Sigma Chemical Company (St. Louis, Mo.), and Boerhinger Mannheim (Indianapolis, Ind.). Other retinoids which are useful herein are described in U.S. Pat. Nos. 4,677,120, issued Jun. 30, 1987 to Parish et al.; 4,885,311, issued Dec. 5, 1989 to Parish et al.; 5,049,584, issued Sep. 17, 1991 to Purcell et al.; 5,124,356, issued Jun. 23, 1992 to Purcell et al.; and Reissue 34,075, issued Sep. 22, 1992 to Purcell et al.. Other suitable retinoids are tocopheryl-retinoate [tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthiochroman-6-yl)-ethynyl]nicotinate). Preferred retinoids are retinol, retinyl palmitate, retinyl acetate, retinyl propionate, retinal and combinations thereof.

(c) Hydroxy Acids

The compositions of the present invention may contain a safe and effective amount of a Hydroxy Acid. Preferred hydroxy acids for use in the compositions of the present invention include salicylic acid and salicylic acid derivatives. When present in the compositions of the present invention, the hydroxy acid is preferably used in an amount of from about 0.01% to about 50%, more preferably from about 0.1% to about 10%, and still more preferably from about 0.5% to about 2%.

Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

Suitable dipeptides for use herein include Carnosine (beta-ala-his). Suitable tripeptides for use herein include, gly-his-lys, arg-lys-arg, his-gly-gly. Preferred tripeptides and derivatives thereof include palmitoyl-gly-his-lys, which may be purchased as Biopeptide CL® (100 ppm of palmitoyl-gly-his-lys commercially available from Sederma, France); Peptide CK (arg-lys-arg); Peptide CK+ (ac-arg-lys-arg-NH₂); and a copper derivative of his-gly-gly sold commercially as lamin, from Sigma (St.Louis, Mo.). Suitable tetrapeptides for use herein include Peptide E, arg-ser-arg-lys. Suitable pentapeptides for use herein include lys-thr-thr-lys-ser. A preferred commercially available pentapeptide derivative composition is Matrixyl®, which contains 100 ppm palmitoyl-lys-thr-thr-lys-ser, commercially available from Sederma, France). Preferably, the peptide is selected from palmitoyl-lys-thr-thr-lys-ser, palmitoyl-gly-his-lys, their derivatives, and combinations thereof.

Anti-Oxidants/Radical Scavengers

The compositions of the present invention may include a safe and effective amount of an anti-oxidant/radical scavenger, preferably anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl glucoside, ascorbyl sorbate), tocotrienols, tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox® ), gallic acid and its alkyl esters, especially propyl gallate, sorbic acid and its salts, lipoic acid, amines (e.g., N,N-diethylhydroxylamine, amino-guanidine), sulfhydryl compounds (e.g., glutathione), dihydroxy fumaric acid and its salts, bioflavonoids, curcumin, lysine, methionine, proline, superoxide dismutase, silymarin, tea extracts, grape skin/seed extracts, melanin, and rosemary extracts may be used. Preferred anti-oxidants/radical scavengers are selected from tocopherol sorbate, tocopherol acetate, other esters of tocopherol, and mixtures thereof. Tocopherol acetate is especially preferred.

Chelators

The compositions of the present invention may contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Nov. 31, 1995. Preferred chelators useful in compositions of the subject invention are furildioxime, furilmonoxime, and derivatives thereof.

Flavonoids

The compositions of the present invention may optionally contain a flavonoid compound. Flavonoids are broadly disclosed in U.S. Pat. Nos. 5,686,082 and 5,686,367. Non-limiting examples of flavonoids useful herein include unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), and mixtures thereof..

When present, the flavonoid compounds are preferably present in concentrations of from about 0.01% to about 20%, more preferably from about 0.1% to about 10%, by weight of the

Anti-Inflammatory Agents

A safe and effective amount of an anti-inflammatory agent may be added to the compositions of the present invention, from about 0.1% to about 10%, alternatively from about 0.5% to about 5%, of the composition.

Nonlimiting examples of “natural” anti-inflammatory agents that are useful herein include candelilla wax, bisabolol (e.g., alpha bisabolol), aloe vera, plant sterols (e.g., phytosterol), and mixtures thereof.

Additional anti-inflammatory agents useful herein include glycyrrhizinate compounds such as dipotassium glycyrrhizinate. A safe and effective amount of an anti-inflammatory agent may be added to the compositions of the present invention, preferably from about 0.1% to about 10%, more preferably from about 0.5% to about 5%, of the composition.

Anti-Cellulite Agents

The compositions of the present invention may contain a safe and effective amount of an anti-cellulite agent. Suitable agents may include, but are not limited to, xanthine compounds (e.g., caffeine, theophylline, theobromine, and aminophylline).

Topical Anesthetics

The compositions of the present invention may contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Tanning Actives

The compositions of the present invention may contain a safe and effective amount of a tanning active, preferably from about 0.1% to about 20% of dihydroxyacetone as an artificial tanning active.

Dihydroxyacetone, which is also known as DHA or 1,3-dihydroxy-2-propanone, is a white to off-white, crystalline powder.

Skin Lightening Agents

The compositions of the present invention may contain a skin lightening agent. When used, the compositions preferably contain from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, ascorbic acid and derivatives thereof (e.g., magnesium ascorbyl phosphate or sodium ascorbyl phosphate), and extracts (e.g., mulberry extract, placental extract). Skin lightening agents suitable for use herein also include those described in the PCT publication No. 95/34280, in the name of Hillebrand, corresponding to PCT Application No. U.S. 95/07432, filed Jun. 12, 1995; and co-pending U.S. patent application Ser. No. 08/390,152 filed in the names of Kvalnes, Mitchell A. DeLong, Barton J. Bradbury, Curtis B. Motley, and John D. Carter, corresponding to PCT Publication No. 95/23780, published Sep. 8, 1995.

Skin Soothing and Skin Healing Actives

A safe and effective amount of a skin soothing or skin healing active may be added to the present composition, preferably, from about 0.1% to about 30%, more preferably from about 0.5% to about 20%, still more preferably from about 0.5% to about 10%, by weight of the composition formed. Skin soothing or skin healing actives suitable for use herein include panthenoic acid derivatives (including panthenol, dexpanthenol, ethyl panthenol), aloe vera, allantoin, bisabolol, and dipotassium glycyrrhizinate.

Antimicrobial and Antifungal Actives

The compositions of the present invention may contain an antimicrobial or antifungal active. A safe and effective amount of an antimicrobial or antifungal active may be added to the present compositions, preferably, from about 0.001% to about 10%, more preferably from about 0.01% to about 5%, and still more preferably from about 0.05% to about 2%.

Examples of antimicrobial and antifungal actives include phenoxyethanol, zinc erythromycin, chlorhexidine gluconate.

Sunscreen Actives

Exposure to ultraviolet light can result in excessive scaling and texture changes of the stratum corneum. Therefore, the compositions of the subject invention may contain a safe and effective amount of a sunscreen active. As used herein, “sunscreen active” includes both sunscreen agents and physical sunblocks. Suitable sunscreen actives may be organic or inorganic.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the composition.

A wide variety of conventional organic sunscreen actives are suitable for use herein. Sagarin, et al., at Chapter VIII, pages 189 et seq., of Cosmetics Science and Technology (1972), and Steinberg, Vol 111 pages 77 et seq., of Cosmetics and Toiletries (1996) discloses numerous suitable actives. Nonlimiting examples of organic sunscreen actives useful herein include octylsalicylate, 2-Phenylbenzimidazole-5-sulphonic acid salts, Salts of Terephthalylidene Dicamphor sulfonic acid, octocrylene, octylmethoxycinnamate, avobenzone, and mixtures thereof.

A safe and effective amount of the organic sunscreen active is used, typically from about 1% to about 20%, more typically from about 2% to about 10% by weight of the composition. Exact amounts will vary depending upon the sunscreen or sunscreens chosen and the desired Sun Protection Factor (SPF).

Particulate Material

The compositions of the present invention may contain a safe and effective amount of a particulate material, preferably a metallic oxide. These particulates can be coated or uncoated, charged or uncharged. Charged particulate materials are disclosed in U.S. Pat. No. 5,997,887, to Ha, et al., incorporated herein by reference. Particulate materials useful herein include; bismuth oxychloride, iron oxide, mica, mica treated with barium sulfate and TiO2, silica, nylon, polyethylene, talc, styrene, polypropylene, ethylene/acrylic acid copolymer, polymethylsilsesquioxane, titanium dioxide, iron oxide, bismuth oxychloride, sericite, aluminum oxide, silicone resin, barium sulfate, calcium carbonate, cellulose acetate, polymethyl methacrylate, and mixtures thereof.

One example of a suitable particulate material contains the material available from U.S. Cosmetics (TRONOX TiO2 series, SAT-T CR837, a rutile TiO2). Typically, particulate materials are present in the composition in levels of from about 0.01% to about 2%, alternatively from about 0.05% to about 1.5%, and from about 0.1% to about 1%, by weight of the composition.

Conditioning Agents

The compositions of the present invention may contain a safe and effective amount of a conditioning agent selected from humectants, moisturizers, or skin conditioners. A variety of these materials can be employed and each can be present at a level of from about 0.01% to about 20%, more preferably from about 0.1% to about 10%, and still more preferably from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose, glucosamine); hyaluronic acid; lactamide monoethanolamine; acetamide monoethanolamine; panthenol; allantoin; and mixtures thereof. Also useful herein are the propoxylated glycerols described in U.S. Pat. No. 4,976,953, to Orr et al, issued Dec. 11, 1990.

Also useful are various C₁-C₃₀ monoesters and polyesters of sugars and related materials. These esters are derived from a sugar or polyol moiety and one or more carboxylic acid moieties.

When the conditioning agent is an emollient it is generally selected from hydrocarbons, fatty acids, fatty alcohols and esters. Isononyl isononanoate is one such hydrocarbon type of emollient conditioning agent. Other hydrocarbons that may be employed include mineral oil, polyolefins such as polydecene, and paraffins such as isohexadecane (e.g. Permethyl 99 Registered TM and Permethyl 101 Registered TM).

Preferably, the conditioning agent is selected from sucrose polyester, panthenol, dexpanthenol, allantoin, and combinations thereof.

Thickening Agent (Including Thickeners and Gelling Agents)

The compositions of the present invention may contain a safe and effective amount of one or more thickening agents, preferably from about 0.1% to about 5%, more preferably from about 0.1% to about 4%, and still more preferably from about 0.25% to about 3%, by weight of the composition.

Classes of thickening agents include the following:

a) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include copolymers of C₁₀-₃₀ alkyl acrylates with one or more monomers of acrylic acid, methacrylic acid, or one of their short chain (i.e., C₁-₄alcohol) esters, wherein the crosslinking agent is an allyl ether of sucrose or pentaerytritol. These copolymers are known as acrylates/C₁₀-₃₀ alkyl acrylate crosspolymers and are commercially available as Carbopol® 1342, Carbopol® 1382, Pemulen TR-1, and Pemulen TR-2, from B.F. Goodrich. Examples of carboxylic acid polymer thickeners useful herein are those selected from carbomers, acrylates/C₁₀-C₃₀ alkyl acrylate crosspolymers, and mixtures thereof.

b) Crosslinked Polyacrylate Polymers

The compositions of the present invention may contain a safe and effective amount of crosslinked polyacrylate polymers useful as thickeners or gelling agents including both cationic and nonionic polymers, with the cationics being generally preferred. Examples of useful crosslinked nonionic polyacrylate polymers and crosslinked cationic polyacrylate polymers are those described in U.S. Pat. No. 5,100,660, to Hawe et al, issued Mar. 31, 1992; U.S. Pat. No. 4,849,484, to Heard, issued Jul. 18, 1989; U.S. Pat. No. 4,835,206, to Farrar et al, issued May 30, 1989; U.S. Pat. No. 4,628,078 to Glover et al issued Dec. 9, 1986; U.S. Pat. No. 4,599,379 to Flesher et al issued Jul. 8, 1986; and EP 228,868, to Farrar et al, published Jul. 15, 1987.

c) Polyacrylamide Polymers

The compositions of the present invention may contain a safe and effective amount of polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

d) Polysaccharides

A wide variety of polysaccharides are useful herein. “Polysaccharides” refer to gelling agents which contain a backbone of repeating sugar (i.e., carbohydrate) units. Examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del.).

Other useful polysaccharides include scleroglucans which are a linear chain of (1-3) linked glucose units with a (1-6) linked glucose every three units, a commercially available example of which is Clearogel™ CS 11 from Michel Mercier Products Inc. (Mountainside, N.J.).

e) Gums

Other thickening and gelling agents useful herein include materials which are primarily derived from natural sources. Examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Compositions of the present invention include a thickening agent selected from carboxylic acid polymers, crosslinked polyacrylate polymers, polyacrylamide polymers, and mixtures thereof, including those selected from carboxylic acid polymers, polyacrylamide polymers, and mixtures thereof.

Composition Preparation

The compositions useful for the methods of the present invention are generally prepared by conventional methods such as are known in the art of making topical compositions. Such methods typically involve mixing of the ingredients in one or more steps to a relatively uniform state, with or without heating, cooling, application of vacuum, and the like.

Methods for Regulating Skin Condition

The skin care kits of the present invention are useful for regulating mammalian skin condition. Such regulation of keratinous tissue conditions can include prophylactic and therapeutic regulation. For example, such regulating methods are directed to thickening keratinous tissue (i.e., building the epidermis and/or dermis layers of the skin and where applicable the keratinous layers of the nail and hair shaft) and preventing and/or retarding atrophy of mammalian skin, preventing and/or retarding the appearance of spider vessels and/or red blotchiness on mammalian skin, preventing and/or retarding the appearance of dark circles under the eye of a mammal, preventing and/or retarding sallowness of mammalian skin, preventing and/or retarding sagging of mammalian skin, softening and/or smoothing lips, hair and nails of a mammal, preventing and/or relieving itch of mammalian skin, regulating skin texture (e.g. wrinkles and fine lines), and improving skin color (e.g. redness, freckles).

Regulating keratinous tissue condition involves topically applying to the keratinous tissue a safe and effective amount of a composition of the present invention. The amount of the composition which is applied, the frequency of application and the period of use will vary widely depending upon the level of skin care actives and/or other components of a given composition and the level of regulation desired, e.g., in light of the level of keratinous tissue damage present or expected to occur.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic application continue throughout the subject's lifetime. Typically applications would be on the order of about once per day over such extended periods, however application rates can vary from about once per week up to about three times per day or more.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are given in CTFA name.

### Examples 1-7

### Water-in-Silicone Skin Cream

Water-in-silicone skin creams are prepared by conventional methods from the following components. Amounts of ingredients are listed in percent by weight of the composition.

| | Ingredient | 1 | 2 | 3 | 4 | 5 | 6 | 7 |
|---|---|---|---|---|---|---|---|---|
| PHASE A: | Water | qs | qs | qs | qs | qs | qs | qs |
| | Disodium EDTA | 0.10 | 0.10 | 0.10 | 0.10 | 0.10 | 0.10 | 0.10 |
| | Methyl Paraben | 0.10 | 0.10 | 0.10 | 0.10 | 0.10 | 0.10 | 0.10 |
| | Propyl Paraben | 0.10 | 0.10 | 0.10 | 0.10 | 0.10 | 0.10 | 0.10 |
| | Niacinamide | 2.0 | 4.0 | 7.5 | 5.0 | 3.50 | 10.00 | 5.0 |
| | Dexpanthenol | 1.0 | 0.50 | 1.0 | 1.0 | 0.50 | 1.0 | 0.50 |
| | Allantoin | 0.2 | 0.2 | | 0.2 | | 0.2 | 0.2 |
| | Benzyl Alcohol | 0.25 | 0.25 | 0.25 | 0.25 | 0.25 | 0.25 | 0.25 |
| | Green Tea Extract | 1.00 | 1.00 | 1.00 | 1.00 | 1.00 | 1.00 | |
| | Glycerin | 9.0 | 11.0 | 20.00 | 10.00 | 7.00 | 15.00 | 15 |
| | Terephthalylidene | | | | | | | 5.0 |
| | dicamphor sulfonic acid¹ | | | | | | | |
| | Palmitoyl Lys Thr Thr Lys | 0.0001 | 0.0002 | | | 0.0003 | | |
| | Ser² | | | | | | | |
| PHASE B: | Dow Coming 9040³ | 8.50 | 14.0 | | 20.00 | 10.00 | 7.50 | 10 |
| | KSG-21⁴ | 2.25 | 2.75 | 15.00 | | 7.00 | 0.50 | 10 |
| | Cyclomethicone | 18 | 25 | 20.00 | 25.00 | 20.00 | 3.00 | 20 |
| | Abil EM-97) | 0.50 | 0.55 | 1.0 | 1.5 | 2.0 | 2.00 | |
| | Vitamin E Acetate | 0.5 | 0.50 | 0.5 | | 0.50 | 0.50 | |
| | Titanium Dioxide | 0.5 | | 0.50 | | | | |
| | GLW75CAP-MP⁵ | | | | | | | |
| | Fragrance | 0.20 | | 0.20 | 0.20 | | 0.20 | |
| | Farnesol | 0.5 | 1.00 | | | | 1.00 | |
| | Parsol 1789⁶ | | | | | 3.00 | 2.00 | |
| | Fytosterol-85⁷ | | | | | 1.00 | | |
| | Octyl Salicylate | | | | | | 5.00 | |
| | Isopropyl Palmitate | | | | | 7.00 | 6.00 | |
| | EA-209⁸ | 2.5 | | | | | | |
| | Tospearl 2000 | | 7.00 | | 1.00 | | | |
| PHASE C | Finsolv TN | | | 2.00 | 2.00 | 2.00 | | |
| | Retinol | | | 0.10 | | | | |
| | Retinyl Propionate | | | | 0.20 | 0.20 | | |

¹Can be obtained from Chimex as Mexoryl SX
²Peptide can be obtained from Sederma
³12% Dimethicone/Vinyl Dimethicone crosspolymer in cyclomethicone from Dow Coming
⁴Available from Shin-Etsu; 25% Dimethicone/Copolyol Crosspolymer in dimethicone
⁵Titanium Dioxide GLW75CAP-MP can be obtained from KOBO
⁶Parsol 1789 can be obtained from Roche
⁷Fytosterol-85 can be obtained from Dragoco
⁸EA-209 can be obtained from KOBO

The ingredients of Phase A are mixed together in a suitable container and the ingredients of Phase B are mixed together in a separate suitable container, both using a suitable mixer (e.g., Tekmar model RW20DZM) equipped with a propeller blade. If Phase C ingredients are present, such ingredients are mixed together in a separate suitable container (where necessary) and are added to Phase B. When both Phases are homogenous, Phase A is slowly added to Phase B while mixing Phase B with propeller blade. Mixing is maintained until the batch is uniform. The resulting emulsion is then milled using a suitable mill (e.g. Tekmar T25) for several minutes until uniform. The product viscosity may be increased to the desired level by additional milling as is understood by one skilled in the art. Once the batch mixture is uniform, the resulting composition is introduced into a suitable dispenser as described herein.

What is claimed is:

1. A skin care kit comprising a water-in-oil emulsion skin care composition contained within a dispensing package that can deliver by full actuation of said package's actuation surface, a predetermined amount of said skin care composition wherein said composition is dispensed onto said actuation surface through openings integrally formed in said actuation surface for applicaton of said composition to the skin.

2. The skin care kit of claim 1 wherein said openings comprise a plurality of upper valve dispensing orifices randomly located about the surface area of the actuation surface.

3. The skin care kit of claim 1 wherein the openings comprise a singular upper valve dispensing orifice.

4. The skin care kit of claim 3 wherein the upper valve dispending orifice is located on the medium point on the diametric axis of the outside diameter of said actuation surface.

5. The skin care kit of claim 1 wherein the amount of said skin care composition delivered when the actuation surface is completely actuated is from about 0.75 ml. to about 1.25 ml.

6. The skin care kit of claim 1 wherein said water-in-oil emulsion comprises:
i) a silicone elastomer;
ii) a continuous hydrophobic phase; and
iii) a discontinuous hydrophilic phase.

7. The skin care kit of claim 6 wherein the composition comprises from about 5% to about 99%, by weight of the composition, of the silicone oil and wherein the silicone oil is present in the hydrophobic phase.

8. The skin care kit of claim 7, wherein the silicone oil is selected from the group consisting of dimethicone, cylcomethicone, and mixtures thereof.

9. The skin care kit of claim 6 wherein the silicone elastomer is selected from the group consisting of emulsifying silicone elastomers, non-emulsifying silicone elastomers, and mixtures thereof.

10. The skin care kit of claim 9, wherein the composition comprises from about 0.1% to about 30%, by weight of the composition, of the silicone elastomer.

11. The skin care kit of claim 6 wherein said discontinuous hydrophilic phase comprises water.

12. The skin care kit of claim 9 wherein said silicone elastomer is an emulsifying elastomer selected from the group consisting of dimethicone copolyol cross polymers and the silicone oil is dimethicone.

13. The skin care kit of claim 9 wherein said non-emulsifying silicone elastomer is selected from the group consisting of dimethicone/vinyl dimethicone cross polymers, and mixtures thereof.

14. The skin care kit of claim 1 wherein the composition further comprises from about 0.1% to about 50% of an additional skin care active selected from the group consisting of vitamins and pro-vitamins, peptides and derivatives thereof, allantoin, particulates, sunscreens, desquamation agents, anti-oxidants, free radical scavengers, chelators, flavanoids, anti-inflammatories, anti-cellulite agents, topical anesthetics, tanning actives, skin lightening agents, anti-microbial actives, anti-fungal actives, conditioning agents, and mixtures thereof.

15. A skin care kit comprising a water-in-oil emulsion skin care composition contained within a dispensing package that can deliver by full actuation of said package's actuation surface, a predetermined amount of said skin care composition wherein said skin care composition is dispensed onto said actuation surface through openings integrally formed in said actuation surface for application of said composition to the skin wherein said skin care kit comprises:
A) a water-in-oil emulsion which comprises:
i) a silicone elastomer;
ii) a continuous hydrophobic phase; and
iii) a discontinuous hydrophilic phase; and
B) the dispenser comprises a container for storing a supply of the skin care composition to be dispensed, said container having a bottom portion and an upper portion, said bottom portion having a slidable follower piston and said upper portion having a pump for dispensing the skin care composition, said pump comprising:
i) a first non-return valve provided in a lower partition wall of the pump unit for controlling communication between the interior of the container and a pump chamber through a first opening formed in said partition wall;
ii) a guide sleeve arrangement surrounding said first opening and having first non-return valve connected thereto, said guide sleeve arrangement extending upwardly from said partition wall to define circumferentially said pump chamber, said guide sleeve arrangement having inner and outer circumferential guide sleeves;
iii) a cup-shaped actuation surface having a peripheral downwardly projecting outer wall portion and an inner tubular section, the latter forming a snap connection to the pressure piston where:
a) said outer wall portion of the actuation surface is slidably engaged with the outer circumferential guide sleeve of the guide sleeve arrangement and the outer wall of the retention ring, being provided with co-operating stop projections to limit axial upward movement of the actuation surface;
b) said outer guide sleeve being integral with said lower partition wall of said pump unit, said tubular section of the actuator surface having a downwardly extending portion having a diameter greater than that of the dispensing orifice and supporting a pressure piston to keep the piston slidably engaged with said inner circumferential guide sleeve of said guide sleeve arrangement, thereby defining a space forming the pump chamber,
d) said dispensing piston having an opening in register with the tubular section of the actuation surface, and
iv) a pressure spring extending between a stationary portion of the lower wall of the retention ring and the actuation surface to bias said actuation surface into a rest position.

16. A skin care kit comprising a water-in-oil emulsion skin care composition contained within a dispensing package that can deliver by full actuation of said package's actuation surface, a predetermined amount of said skin care composition wherein said skin care composition is dispensed onto said actuation surface through openings integrally formed in said actuation surface for application of said composition to the skin wherein said skin care kit comprises:
A) a water-in-oil emulsion which comprises:
j) a silicone elastomer;
ii) a continuous hydrophobic phase; and
iii) a discontinuous hydrophilic phase; and
B) the dispenser comprises a container for storing a supply of the skin care composition to be dispensed, said container having a bottom portion and an upper portion, said bottom portion having a slidable follower piston and said upper portion having a pump for dispensing the skin care composition, said pump comprising:
i) a first non-return valve provided in a lower partition wall of the pump unit for controlling communication between the interior of the container and a pump chamber through a first opening formed in said partition wall;
ii) a guide sleeve arrangement surrounding said first opening and said guide sleeve arrangement extending upwardly from said partition wall to define circumferentially said pump chamber, said guide sleeve arrangement having inner and outer circumferential guide sleeves;
iii) a cup-shaped actuation surface having a peripheral downwardly projecting outer wall portion and an inner tubular section, the latter forming a snap connection to the spring housing where:
a) said outer wall portion of the actuation surface is slidably engaged with the outer wall of the retention ring;
b) said spring housing is slidably engaged with an inner guide sleeve being integral with said retention ring, said spring housing being provided with a co-operating stop projection engaged to said inner guide sleeve to limit axial upward movement of the actuation surface; spring housing having a tubular section engaged internally to said actuation surface and externally to the pressure piston;
c) said tubular section of the actuation surface having a downwardly extending portion having a diameter greater than that of the dispensing orifice;
d) said spring housing forming a snap connection to pressure piston to keep the piston slidably engaged with an inner circumferential guide sleeve of said guide sleeve arrangement, thereby defining a space forming the pump chamber;
e) said pressure piston having an opening in register with the tubular section of the actuation surface and spring housing;
f) a second non-return valve provided in the tubular portion of the pressure piston of the pump unit for controlling communication between the pump chamber and tubular portion of the actuation surface leading to the orifice through a second opening formed in said pressure piston; and
iv) a pressure spring extending between a stationary portion of the lower wall of the retention ring and the spring housing to bias said actuation surface into an upward rest position.

17. The skin care kit of claim 16 wherein said openings comprise a plurality of upper dispensing orifices randomly located about the surface area of the actuation surface.

18. The skin care kit of claim 17 wherein the openings comprise a singular upper dispensing orifice wherein said upper dispensing orifice is located on the medium point on the diametric axis of the outside diameter of said actuation surface.

19. The kit according to claim 16 wherein said skin care composition comprises from about 0.1% to about 30%, by weight of the composition, of the silicone elastomer.

20. The skin care kit of claim 16 wherein said composition further comprises from about 0.1% to about 50% of an additional skin care active selected from the group consisting of vitamins and pro-vitamins, peptides and derivatives thereof, allantoin, particulates, sunscreens, desquamation agents, anti-oxidants, free radical scavengers, chelators, flavanoids, anti-inflammatories, anti-cellulite agents, topical anesthetics, tanning actives, skin lightening agents, anti-microbial actives, anti-fungal actives, conditioning agents, and mixtures thereof.
